(19) United States
(12) Patent Application Publication
(10) Pub. No.: US 2006/0165810 A1
(43) Pub. Date: Jul. 27, 2006

(54) Controlled release from block co-polymer worm micelles

(75) Inventors: Dennis Discher, Philadelphia, PA (US); Yan Geng, Langhorne, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Trustees of the University of Pennsylvania

(21) Appl. No.: 11/320,198

(22) Filed: Dec. 28, 2005

Related U.S. Application Data
(60) Provisional application No. 60/639,501, filed on Dec. 28, 2004.

(51) Int. Cl. A61K 38/17; A61K 48/00; A61K 9/50; A61K 33/00; A61K 31/56; A61K 9/16

(52) U.S. Cl. 424/490; 424/600; 514/2; 514/44; 514/169

(57) ABSTRACT

Provided is a method of controlling the release of at least one encapsulated active agent from a worm-like micelle, wherein each worm-like micelle comprises one or more amphiphilic block copolymers that self assemble in aqueous solution, without organic solvent or post assembly polymerization; wherein at least one of said amphiphilic molecules is a hydrophilic block copolymer and at least one of said amphiphilic molecules is a hydrophobic block copolymer which is hydrolyticaly unstable in the pH range of about 5 to about 7. The loaded worm-like micelles of the present invention are particularly suited for the stable and controlled transport, delivery and storage of materials, either in vivo or in vitro.

### CROSS REFERENCE TO RELATED APPLICATIONS

This application claims priority to U.S. Provisional Application No. 60/639,501, filed Dec. 28, 2004, the content of which is herein incorporated in its entirety.

### GOVERNMENT SUPPORT

This work was supported in part by grants from the National Science Foundation, grant number NSF-MRSEC, and also by grants from the National Institutes of Health, grant number NIH R21. The government may have certain rights in this invention.

### FIELD OF THE INVENTION

The present invention relates to methods of using worm-like micelles formed from block copolymer amphiphiles as controlled-release delivery vehicles.

### BACKGROUND OF THE INVENTION

Small amphiphiles of natural origin have inspired the engineering of high molecular weight analogs, which also self-assemble in aqueous solution into complex phases in aqueous media. Amphiphilic multiblock copolymers self-assemble in water into various stable morphologies. Synthetic control over the molecular composition permits novel controls over the properties of membranes assembled from super-amphiplhiles (Hajduk et al., J. Phys. Chem. B 102:4269-4276 (1998)).

Diblock copolymers are prepared, for example, using a two-step anionic polymerization procedure (Hillmyer et al., Macromolecules 29:6994-7002 (1996), wherein copolymers are dissolved in chloroform and dried on glass to form a film that is hydrated with water at 50-60° C. In dilute aqueous solutions certain diblock copolymers, such as polyethyleneoxide-polyethylethylene (PEO-PEE, also referred to simply as OE, wherein PEO is structurally equivalent to PEG), have been shown to form unilamellar vesicles and micelles in which polyethyleneoxide-polybutadiene (PEO-PBD, also referred to simply as OB) mesophases were successfully cross-linked into bulk materials with completely different properties, notably an enhanced shear elasticity (Won et al., Science 283:960-963 (1999); Discher et al., Science 284:1143-1146 (1999)). The resulting miicrostructures, termed polymersomes, though assembled in water, can withstand dehydration, as well as exposure to organic solvents, such as chloroform (U.S. patent application Ser. No. 09/460,605), and controlled release of encapsulants from such vesicles was subject to denaturation of the selected blend of block copolymers (Ahmed et al., J. Controlled Release, 96:37-53 (2004); CIP of U.S. patent application Ser. No. 09/460,605, based upon Provisional Appl. 60/459,049).

Block copolymers of both PEG and a hydrolytically susceptible polyester of either polylactic acid (PLA) (Belbella et al., Internat'l J. Pharmaceutics 129:95-102 (1996); Anderson et al., Adv. Drug Delivery Rev. 28:5-24 (1997); Brunner et al., Pharmaceutical Research 16:847-853 (1999); Woo et al., J. Controlled Release 75:307-315 (2001)) or polycaprolactone (PCL) (Pitt in Biodegradable Polymers as Drug Delivery Systems, Langer, Chasin (eds.), Marcel Dekker, New York, N.Y., 1990, pp. 71-120; Chawla et al., Internat'l J. Pharmaceutics 249:127-138 (2002)) have been described (Matsumoto et al., Internat'l J. Pharmaceutics 185:93-101 (1999); Allen et al., J. Controlled Release 63:275-286 (2000); Panagi et al., Internat'l J. Pharmaceutics 221:143-152 (2001); Riley et al., Langmuir 17:3168-3174 (2001); Avgoustakis et al., J. Controlled Release 79:123-135 (2002); Discher et al., Science 297:967-973 (2002a); Meng et al., Macromolecules 36:3004-3006 (2003); Ahmed et al., Langmuir 19:6505-6511 (2003)). Vesicle formulations prepared using hydrolyzable diblock copolymers of polyethyleneglycol-poly-L-lactic acid (PEG-PLA) or polyethyleneglycol-polycaprolactone (PEG-PCL), with or without inert PEG-PBD (Discher et al., supra, 1999)), have been shown to provide programmed control over release kinetics (Ahmed et al., supra, 2004; CIP Patent Appl., supra), based on the general principle of blending degradable and inert copolymers.

Controlled release drug-delivery vehicles run the gamut from self-assemblies of lipids (liposomes) (Gref et al., Science 263:1600-1603 (1994); Lasic et al., Curr. Op. Solid St. M. 3:392 (1996)) to biochemically modified quantum dots (Akerman et al., Proc. Nat'l Acad. Sci. (USA) 99:12617-12621 (2002)). However, all vehicles studied to date have had the same spherical geometry. Spherical liposomes (diameter ˜100 nm) are cleared from the vasculature of small mammals hours after injection (Blume et al., Biochim. Biophys. Acta 1029:91 (1990)), although the polymersomes, assembled from PEG-based copolymers, have shown an increased circulation time compared to liposomes, wherein polymersomes have half-lives of approximately one day in vivo (Photos et al., supra, 2003).

Encapsulation studies have shown loading in the controlled release vesicles to be comparable to liposomes. Rates of release of encapsulants from the hydrolysable vesicles were accelerated by an increased proportion of PEG, but were delayed with more hydrophobic chain chemistry, i.e., PCL. Rates of release rose linearly with the molar ratio of degradable copolymer blended into membranes of a non-degradable, PEG-based block copolymer (PEG-polybutadiene). Thus, poration occurred as the hydrophobic PLA or PCL block was hydrolytically scissioned, progressively generating an increasing number of pore-preferring copolymers in the membrane, which when combined with the phase behavior of the amphiphiles, triggered transition from membrane to micelle kinetics, resulting in controlled release of the encapsulant.

Worm micelles have been formed from small (˜500-1000 g/mol) amphiphiles (Walker, Curr. Opin. Colloid Interface Sci. 6:451 (2001)), but were unstable and quickly fell apart in dilute aqueous concentrations. As a result, until the inventors invention reported in Publ. US Pat. Appl. 2005/0180922, published Aug. 19, 2005 typical surfactant worm micelles could not survive injection as intact aggregates into the circulation of an animal. Nevertheless, although without controlled release, Ruoslahti and coworkers used the micron-long filamentous phage, M13, in a phage display method for identifying ligands that bind to xenoplants of various human cancers (Pasqualini et al., Nature 380:364-366 (1996); Pasqualini et al., Nat. Biotechnol. 15:542-546 (1997)). Once the targeting ligand was identified, it was chemically conjugated to a chemotherapeutic hydrophilic drug (doxorubicin) and successfully used to treat tumors in live animals (Arap et al., Science 279:377 (1998)).

Therefore, despite the success of filamentous phage and polymersome delivery systems, until the present invention there has clearly been a need for novel, stable, aqueously-formed constructs, which can be broadly engineered, but still have the advantageous features of worm micelles necessary to permit controlled-release, biological delivery, including: biocompatibility, selective permeability to solutes, the ability to retain internal aqueous components and control their release, and the ability to deform while remaining relatively tough and resilient. Moreover, such novel constructs must also be able to target selected cells or cell types for the controlled delivery and release of encapsulated contents.

### SUMMARY OF THE INVENTION

The present invention meets the need in the art by providing worm micelles as controlled-release delivery vehicles, particularly drug delivery vehicles, that are prepared from high molecular weight diblock amphiphilic copolymers (e.g., >1-4000 g/mol), which in contrast to early worms prepared from low molecular weight lipids and surfactants, are stable, synthetic, non-living assemblies, even at body temperature (37° C.). The preferred copolymers comprise a hydrophilic PEO (polyethylene oxide) block and one of several hydrophobic blocks that drive self-assembly of worm-like micelles, up to microns in length, in water and other aqueous media. The PEO block of the polymer (which is the same as polyethylene-glycol; PEG) is widely known to make interfaces very biocompatible, thus the worm-like micelles are stable in blood in vitro and in blood flow in vitro and in vivo.

As described in detail by the inventors in Publ. US Pat. Appl. 2005/0180922 (herein incorporated by reference in its entirety for all purposes), visualization of the worm-like micelles can be achieved by fluorescence microscopy after incorporating fluorescent dyes into the micelle cores dyes. Increasing the molecular weight of the copolymers increases both the diameter of the worm-like micelles (from about 10 to 40 nm) and their stiffness. In addition, in the present invention, biotinylated copolymers were blended with pristine copolymers prior to forming micelles by simple hydration of a dried copolymer film.

For drug delivery, the worm micelles of the present invention are shown to be able to incorporate a range of hydrophobic drugs into the cores of the worm-like micelles, and methods are provided to chemically modify the ends of the PEO blocks to make the worm-like micelles specifically bind to suitable surfaces and cells. The present invention, therefore, provides worm micelles which encapsulate one or more “active agents,” which include, without limitation compositions such as a drug, therapeutic compound, dye, nutrient, sugar, vitamin, protein or protein fragment, salt, electrolyte, gene or gene fragment, product of genetic engineering, steroid, adjuvant, biosealant, gas, ferrofluid, or liquid crystal. The thus “loaded” worm micelle may be further used to transport an encapsulatable material (an “encapsulant”) to its surrounding environment.

The present invention provides methods of using the worm micelles to transport one or more of the above identified compositions to a patient in need of such transport activity. For example, the worm micelle could be used to deliver a drug or therapeutic composition to a patient's tissue or blood stream.

Further provided are methods for controlling the release of an encapsulated material from a worm micelle. For example, the worm-like micelles can be fragmented to sub-micron lengths, if desired, and they will flow through nanoporous matrices, including recognized models for brain tissue matrix. Based upon findings using the cytotoxic drug paclitaxel commonly used against cancer cells, further provided is a method of using the worm-like micelles of the present invention to efficiently target and kill cells.

Thus, it is an object of the invention to provide worm micelles for use as drug delivery vehicles, as well as methods for their preparation and for the encapsulation of one or more active agents, and for the controlled release of same. It should be noted that the terms “worm micelle”, “worm-like micelle” and “filomicelles” are used interchangeably herein to mean the same assembly, and are often simply referred to as “lworms” or “micelles.”

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended drawings. It should be understood, however, that the invention is not limited to the precise arrangements and instrumentalities shown.

FIG. 1 illustrates the change in contour length distributions over time in worm micelles having different initial molecular weights.

FIGS. 2a and 2b demonstrate that the PCL in the copolymer hydrolyzes from the end by “chain-end cleavage” to produce 6-HPA rather than by a process of “random scission” that would produce a mixture of various degradation products, such as dimers, trimers, tetramers, and larger oligomers. FIG. 2a illustrates the formation of 6-HPA over time from the hydrolysis of OCL1 worm micelles. As shown, the amount of 6-HPA, the predominant new peak, increased significantly over time, while at the same time, no other predominant peak was observed. FIG. 2b illustrates that the new predominant peak formed by hydrolysis is actually 6-HPA, as opposed to being a dimer, trimer, tetramer or larger caprolactone oligomers, as shown by GPC analysis.

FIG. 3 illustrates the percentage of caprolactone units remaining in worm micelles over time from block copolymers of differing molecular weights by ¹H NMR analysis.

### DETAILED DESCRIPTION OF PREFERRED EMBODIMENTS OF THE INVENTION

The present invention characterizes giant and stable worm-like micelles formed in water from a series of blended and cross-linkable polyethyleneoxide-based (PEO) diblock copolymer amphiphiles that mimic the flexibility of various cytoskeletal filaments, and provide methods for encapsulation and cell-targeted micropore drug delivery. Worm micelles are amphiphilic aggregates poised in size between molecular scale spherical micelles and much larger lamellar structures, such as vesicles, and fluidity and hydrodynamics play important roles in there formation.

The worm micelles of the present invention are formed from synthetic, amphiphilic copolymers. An “amphiphilic” substance is one containing both polar (water-soluble) and hydrophobic (water-insoluble) groups, and polymers are macromolecules comprising connected monomeric units. The monomeric units may be of a single type (homogeneous), or a variety of types (heterogeneous). The physical behavior of the polymer is dictated by several features, including the total molecular weight, the composition of the polymer (e.g., the relative concentrations of different monomers), the chemical identity of each monomeric unit and its interaction with a solvent, and the architecture of the polymer (whether it is single chain or branched chains).

The preferred class of polymer selected to prepare the worm micelles of the present invention is the “block copolymer.” Block copolymers are polymers having at least two, tandem, interconnected regions of differing chemistry. Each region comprises a repeating sequence of monomers. Thus, a “diblock copolymer” comprises two such connected regions (A-B); a “triblock copolymer,” three (A-B-C), etc. Each region may have its own chemical identity and preferences for solvent. Thus, an enormous spectrum of block chemistries is theoretically possible, limited only by the acumen of the synthetic chemist.

In the “melt” (pure polymer), a diblock copolymer may form complex structures as dictated by the interaction between the chemical identities in each segment and the molecular weight. The interaction between chemical groups in each block is given by the mixing parameter or Flory interaction parameter, χ, which provides a measure of the energetic cost of placing a monomer of A next to a monomer of B. Generally, the segregation of polymers into different ordered structures in the melt is controlled by the magnitude of χN, where N is proportional to molecular weight. For example, the tendency to form lamellar phases with block copolymers in the melt increases as χN increases above a threshold value of approximately 10.

A linear diblock copolymer of the form A-B can form a variety of different structures. In either pure solution (the melt) or diluted into a solvent, the relative preferences of the A and B blocks for each other, as well as the solvent (if present) will dictate the ordering of the polymer material, producing the numerous resulting structural phases.

To form a stable membrane in water, the absolute minimum requisite molecular weight for an amphiphile must exceed that of methanol HOCH₃, which is undoubtedly the smallest canonical amphiphile, with one end polar (HO—) and the other end hydrophobic (—CH₃). Copolymer synthesis using anionic polymerization techniques is described by Hillmyer et al., supra, 1996. For film hydration techniques (see, Menger et al., Acc. Chem. Res. 31:798 (1998)). Formation of a stable lamellar phase more precisely requires an amphiphile with a hydrophilic group whose projected area, when viewed along the membrane's normal, is approximately equal to the volume divided by the maximum dimension of the hydrophobic portion of the amphiphile. Therefore, assembly of diblock copolymer amphiphiles into one of the worm micelles depends primarily on the weight fraction (w) of the hydrophilic block relative to the total copolymer molecular weight (Discher et al., supra, 1999; Hajduk et al., supra, 1998; Zhang et al., Science 268:1728 (1995)). Higher w gives predominantly spherical micelles, whereas lower w yields vesicles (Israelachvili, In Intermolecular & Surface Forces, Academic Press: London, 1992, pp 380-382).

For example, using a diblock copolymer comprising a hydrophilic polyethyleneoxide (PEO) block and a hydrocarbon-based hydrophobic block, spontaneously form into vesicles or polymersomes when the bulk copolymer is added to water when the fraction of the PEO block is ˜25-42%. However, with a relatively small increase in the PEO fraction, such that w_{EO}≈45-55%, hydration of the PEO overcomiipenisates, and osmotic force induces a curvature in the aggregate, leading to the assembly of mainly worm micelles. Such aggregate formation is strongly driven by the relatively high molecular weight of the hydrocarbon segment. This creates an interfacial tension, which separates the core from PEO, as well as the bulk aqueous phase. Despite this sensitivity in composition, the worm micelle assemblies of 4000-5000 g/mol diblocks prove exceedingly stable, yet flexible.

Cryo-transmissioni electron microscopy (cryo-TEM) has shown that the worm micelles, made of block copolymers of molecular weight MW ˜4 kDa, have hydrophobic cores of ˜10 nm. Worm micelles assembled from PEO-based nonionic copolymers prove extremely stable in aqueous media, and the dynamics of the worms can be directly visualized with fluorescence microscopy techniques. By blending and polymerizing inert (e.g., OE6) and cross-linkable (e.g., OB3) copolymers, micelles up to tens of microns long (or longer) are formed with sub-micron persistence lengths (1) that continuously span more than 2 orders of magnitude—from submicron (about 500 nm) to submillimeter (about 100 μm), in agreement with estimations from neutron scattering (Won et al., J. Phys. Chem. B 105:8302 (2001)). Under quiescent conditions, the persistence length of the pristine worms is just large enough for the backbone to be clearly visualized when the worm is confined in a gap or a microcapillary.

Although the diameter (d) of the worm micelles is similar to the membrane thickness of polymersomes, the Brownian dynamics of worm micelles are highly pronounced in contrast to the membranes. Autocorrelation analyses of the easily identified end-to-end distance yields relaxation times of seconds for the micron long worms. Although these are fluid assemblies, the stability of the worms is clear, however, and appears fully consistent with the high γ that drives membrane formation and underlies the stability of the micelles.

In a flow field, rather than under quiescent conditions, the fluid worms orient and stretch with DNA-like scaling, and respond in a way roughly in agreement with present theory for polymers under flow (see Publ. US Pat. Appl. 2005/0180922). It is clear, however, that the worms can withstand very high flow fields which can be estimated to impose tensions <1 μN/m.

Worm Flexibility. The two types of worms—fluid or cross-linked—represent the two extremes at either end of a continuous stiffness scale that can be experimentally realized by blending various block copolymers, such as a saturated polyethylethylene (PEE) copolymer with a cross-linkable polybutadiene (PBD) copolymer. The two types of copolymers have already been shown (using membranes) to be fully miscible, and PBD can be successfully reacted to give a range of stabilities and stiffnesses (Discher et al., supra, 2002a). For worms made with similar MW copolymers, there appears an interesting percolation of the rigidity at relatively low mole fractions of PBD (˜20%).

The effect of cross-linking is shown in Publ. US Pat. Appl. 2005/0180922, and adds to it the measured persistence lengths for giant worm-like micelles. For worms with flexibility equal to or greater than OB9, l_{P} is calculated using <R²>=2l_{P}L[1−e^{L/l}_{P}] where R is the end-to-end distance of the worm and L is the contour length. As schematically shown OB class worms can be pristine or fully cross-linked through polymerization, and OE diblocks can be added to dilute the cross-links and decrease worm stiffness. OB19 worms have diameters that are ˜2.5 times larger than OB3 worms.

As shown, worm-like micelles can emulate the bending rigidity of various ubiquitous biopolymers, from intermediate filaments to microtubules, through selection of different sized copolymers and chemical fixation of unsaturated butadiene bonds. While the principles behind blending and cross-linking are increasingly understood, the subtlety in controlling rigidity with worn diameter stems from the hypothesis that molecules in a fluid worm will rearrange and significantly relax any curvature stress. Factors affecting worm stiffness include scaling of /p with worm diameter d, as well as worm branching and spontaneous curvature effects in cross-linking.

There are presently at least two ways to explore for augmenting the bending rigidity of worn micelles given the present chemistry: (1) chemically cross-link the BD blocks in the worm core to create a solid worm-like micelle, and/or (2) increase the diameter of the worm by assembling the worm from larger copolymers. In the first instance, double bonds in the hydrophobic block of PBD allow cross-linking to be introduced by solution free radical polymerization into the worm cores (Won et al., supra, 1999). Worms can thus be made even more stable and solid, emulating a classic covalent polymer chain, but at a more mesoscopic scale.

Free radical cross-linking within OB3 worms increases worm persistence length by more than 100-fold from l_{P}=0.5 μm to a cross-linked value, l_{PX}[100 μm. As used herein, a term followed by an “X” means that it is fully cross-linked, e.g., OB3-X is fully cross-linked OB3. The persistent length of a cross-linked species is referred to as l_{PX}. Moreover, to interpolate both within this range of rigidities and also from fluid to solid states, a PEO-PEE analog of OB3 (OE6) can be blended into the worm in varying concentrations before free radical polymerization of the PBD double bonds.

By combining techniques (1) and (2) above, OB worms of large diameter (up to d=39 nm; Table 1 duplicated from Publ. US Pat. Appl. 2005/0180922) can be fully cross-linked to form almost inflexible solid cylinders (OB19-X) with a persistence length approaching that of a microtubule. The copolymers listed in Table 1 thus span bending rigidities of ubiquitously expressed biopolymers that range from intermediate filaments to microtubules.

TABLE 1
Structural Details of Poly(ethylene oxide)-Polybutadiene (OB) and
Poly(ethylene oxide)-Polyethylethylene (OE) Diblock Copolymers.

| Designation | Polymer formula | Mₙ (kg/mol) | w_{EO} | d(nm)* |
|---|---|---|---|---|
| OE2 | EO₄₄-EE₃₇ | 3.6 | 0.54 | 10.8* |
| OE6 | EO₄₆-EE₃₇ | 4.1 | 0.48 | 12.5* |
| OE7 | EO₄₆-EE₃₇ | 3.9 | 0.45 | 11.4^{†} |
| OB3 | EO₅₅-BD₄₅ | 4.9 | 0.51 | 14.2^{‡} |
| OB9 | EO₅₀-BD₅₄ | 5.2 | 0.43 | 15.7 |
| OB18 | EO₈₀-BD₁₂₅ | 10.4 | 0.35 | 27.0 |
| — | EO₁₀₅-BD₁₇₀ | 14 | 0.34 | 34.0 |
| OB19 | EO₁₅₀-BD₂₅₀ | 20 | 0.33 | 39.0 |

Diameters denoted by (*) were determined by a best-fit of referenced and measured date.
^{†}see, Won et al., J. Phys. Chem. B 106: 3354 (2002);
^{‡}see, Won et al., supra, 1999.
⁺ see, Jain and Bates, Science 300: 460-464 (2003).

Measurements of worm diameter d from cryo-TEM images show a systematic dependence on the length of the hydrophobic chain (Nₕ), which has also been found for membranes (Bermudez et al., Macromolecules 35:8203-8208 (2002)). Fitting a power law to the referenced and measured data in Table 1, produces a curve wherein the diameter of the worms fits best to d=1.38Nₕ^{0.61}. A fully stretched polymer of Nₕ groups would theoretically assemble into an object with diameter, d˜Nₕ¹, whereas ideal random coils, such as in a melt, would give an object with d˜Nₕ^{0.5}. The copolymers studied herein are in the strong segregation limit (SSL) where interfacial tension, γ, balances chain entropy, so that d˜ₕ^{0.67} is expected (Bates, supra, 1991; Bermudez et al., supra, 2002). The scaling exponent obtained of 0.61 is thus slightly closer to the SSL expectations than the scaling found for membranes assembled from a subset of the same copolymers in Table 1.

Moreover, the radius of gyration (Rg) can be calculated using Rg=b(Nₕ/6), where b=0.54 nm has been experimentally determined for the PEO-PEE copolymers (Almdal et al., Macromolecules 35:7685 (2002)). For OE7, for example, Rg=1.3 nm, which indicates that the copolymer is stretched about 4- to 5-fold compared to the worm radices, d/2 (Table 1). This result is fully consistent with strong lateral squeezing of chain configurations by interfacial tension that extends the chain into the core and thus forms the basis for SSL theory. Thus, while scaling of d with Nₕ alone (d˜Nₕ^{0.61}) is less convincing of SSL versus a simpler melt (d˜Nₕ^{0.5}), the strong stretching is indicative of the SSL.

Given the wide range of core diameters, d, for the worms in Table 1, the scaling relation for the worm persistence length, l_{P}, can be experimentally determined. Dimensional analysis shows that a fluid cylinder whose rigidity is dominated by γ has a persistence length that scales with core diameter in the form l_{P}=φγd³/k_{B}T, where φ is a constant. Based on extensive measurements of membrane elasticity, γ is already known to be a single constant for the OB and OE series of copolymers (Bermudez et al., supra, 2002). Conversely, a solid rod or cylinder, also of diameter d, follows the classical beam theory scaling of l_{P}˜d (Cornelissen et al., supra, 1998; Landau et al., Theory of Elasticity, 3^{rd} ed., Butterworth-Heinemann: Oxford, 1986, chap. 2), where the energy scale for a beam is set by an elastic constant (E) for the core, rather than by γ. When fit by power laws (best fit), the data shows that bending rigidities of the worm micelles have a scaling exponent of 2.8 (l_{P}˜d^{2.8}). Despite chain entanglement in the core, which could effectively solidify it, the scaling result more closely follows the cubic scaling behavior of classical fluid assemblies of lipid-size amphiphiles (˜d 3), rather than solid-core cylinders, rods or beams (˜d 4) (A=0.0004, A₃=0.00023, A₄=9×10⁻⁶) Therefore, given this exponent and γ=25 pN/nm, then φ=1/20 in l_{P}=φγd^{2.8}/k_{B}T. As a result, by polymerizing the unsaturated bonds of assembled copolymers, fluid worms are clearly converted to solid-core worms, extending the bending rigidity from that of intermediate filament biopolymers to actin filaments and, in principle, microtubules.

Cross-linking percolation and spontaneous curvature. As noted above, cross-linked blends of PEO-PBD and PEO-PEE copolymers form worms that span bending rigidities between the fluid PEO-PEE worm (or pristine PEO-PBD worm) and the fully cross-linked, solid PEO-PBD worm. Through partial cross-linking, polymerized worms are further shown to lock in spontaneous curvature at a novel fluid-to-solid percolation point.

The stability, loading capacity, and stealthiness of these superpolymer aggregates make them ideal assemblies for addressing questions of dynamics concerning polymeric objects, such as internal vs. external viscosity effects and collective rheology of synthetic and biological systems. They also establish a foundation for focused material applications and demonstrate their utility for flow-intensive delivery applications, such as phage-mimetic drug carriers and micropore delivery, and for the creation of synthetic cytoskeletons or other structures.

Biocompatibility, encapsulation and use for delivery of active agent(s). Because of the perselectivity of the bilayer, materials may be “encapsulated” by intercalation into the hydrophobic membrane core of the worm micelle of the present invention, resulting in a “loaded” worm micelle. The term “loaded” also refers to the association of materials with the worm micelle. Numerous technologies can be developed from such micelles, owing to the numerous unique features of the bilayer and the broad availability of super-amphipliles, such as diblock, triblock, or other multi-block copolymers.

The synthetic micelle membrane can exchange material with the “bulk,” i.e., the solution surrounding the micelles. Each component in the bulk has a partition coefficient, meaning it has a certain probability of staying in the bulk, as well as a probability of remaining in the membrane. Conditions can be predetermined so that the partition coefficient of a selected type of molecule will be much higher within the membrane of a micelle, thereby permitting the worm micelle to decrease the concentration of a molecule, such as cholesterol, in the bulk. In the alternative, worm micelles can be formed with a selected molecule, such as a hormone, incorporated within the membrane, so that by controlling the partition coefficient, the molecule will be released into the bulk when the micelle arrives at a destination having a higher partition coefficient.

By “biocompatible” is meant a substance or composition that can be introduced into an animal, particularly into a human, without significant adverse effect. For example, when a material, substance or composition of matter is brought into a contact with a viable white blood cell, if the material, substance or composition of matter is toxic, reactive or biologically incompatible, the cells will perceive the material as foreign, harmful or immunogenic, causing activation of the immune response, and resulting in immediate, visible morphological changes in the cell. A “significant” adverse effect would be one that is considered sufficiently deleterious as to preclude introducing a substance into the patient.

An enormously wide range of hydrophilic or hydrophobic materials can be associated with or encapsulated within a worm micelle, e.g., proteins and proteinaceous compositions and other carriers for drugs, therapeutics and other biomaterials, as well as marker preparations. Such an encapsulated material is also referred to herein as an “encapsulant” or “active agent.” Encapsulation applications range, without limitation from, e.g., drug delivery (aqueous insoluble drugs), to optical detectors (fluorescent dyes), to the storage of oxygen, and the like.

A variety of fluorescent dyes of the type that can be incorporated within the worm micelles could include small molecular weight fluorophores, such as rhodamine. Imaging of the fluorescent core can be accomplished by standard fluorescent videomicroscopy. Permeability of the micelles to the fluorophore can be measured by manipulating the fluorescently-filled micelles, and monitoring the retention of fluorescence against a measure of time.

It is clear from the foregoing and from the Example that follows, that worm micelles are particularly useful for the controlled transport (e.g., controlled delivery to the immediately surrounding environment) of hormones, proteins, peptides or polypeptides, sugars or other nutrients, drugs, medicaments or therapeutics, including genetic therapeutics, steroids, vitamins, minerals, salts or electrolytes, genes, gene fragments or products of genetic engineering, dyes, adjuvants, biosealants and the like. In fact, the morphology of the worm micelles may prove particularly suited to the targeted delivery and controlled release of biocompatible compounds to a patient. They are ideal for intravital drug delivery because they are biocompatible; that is they contain no organic solvent residue and are made of nontoxic materials that are compatible with biological cells and tissues. Thus, because they can interact with plant or animal tissues without deleterious immunological effects, any drug deliverable to a patient could be incorporated into a biocompatible worm micelle for delivery.

While drug delivery by worm micelles is known, as disclosed for example in Publ. US Pat. Appl. 2005/0180922, and the worm micelles have been shown to be far more stable than previously known vesicles, methods of controlling the release of the active agent from the worm micelle were unknown until the present invention. The rate and extent of release of the encapsulated active agent from the worm micelle carrier is controlled by the rate and degree of hydrolysis of the copolymers in the worm micelles. The rate of hydrolysis is determined by the chemical composition of the copolymers as well as the pH and temperature of the environment around the worm micelles. The chain length of the worm micelle shortens through chain-end hydrolysis of the hydrophobic co-polymer in the micelle until the preferred morphology of the micelle shifts from a wormlike cylinder, to a sphere. The rate of chain length shortening is controlled by selecting the ratio of the hydrolytically degradable hydrophobic copolymer to the hydrophilic copolymer when forming the wor micelles.

The visualization and characterization, including stability, flexibility, and persistence length (wherein l_{P}=κ/k_{B}T, where κ is bending rigidity) of the self-assembled and highly stable worm micelles in aqueous solution, have been examined in Publ. Pat. Appl. 2005/0180922 using two worm-forming diblocks—one with an inert hydrophobic block of PEE (polyethylethylene), designated OE6, and another with cross-linkable PBD (polybutadiene), designated OB3. The methods of micelle preparation disclosed in the 2005/0180922 application are also particularly preferred in the present invention because the vesicle preparation is without the use of co-solvent. Any organic solvent used during the disclosed synthesis or film fabrication method has been completely removed before the actual vesicle formation. Therefore, the worm micelles of the present invention are free of organic solvents, distinguishing the worm micelles from the prior art and making them uniquely suited for bio-applications. By blending and polymerizing inert and cross-linkable copolymers, the resulting micelles were up to tens of microns long with persistence lengths that continuously spanned more than 2 orders of magnitude from submicron to submillimeter. The complete and extraordinarily efficient cross-linking of this system has been verified elsewhere by testing the chloroform extractability of copolymer (Won et al., supra, 1999).

Importantly, the worms were shown in Publ. US Pat. Appl. 2005/0180922 to operate as a stable aggregate that did not fragment under flow-imposed tensions that were estimated to reach ˜1 μN/m. Tension on worms was calculated from a plane Poiseuille flow model with a velocity profile vₓ(y)=3v[1−(y/H)²], wherein v is the average flow velocity, y is the distance between coverslips, and H is the gap height. The tension is the shear stress (μ∂vₓ/∂y; μ is viscosity) integrated over the contour length of each worm.

In light of the stability, flexibility and convective responsiveness of the worm micelles, Publ. US Pat. Appl. 2005/0180922 relied upon principles of in vitro targeting and in vivo circulation to examine the ability of the worms to target and deliver hydrophobic drugs to a host cell (e.g., the ability to bind to cells and transfer encapsulated contents) of the worm micelles using biotin (which is also a vitamin) and a small ligand that binds to a receptor that is generally upregulated on tumor cells. Length distributions of the biotinylated worm micelles (25% biotin copolymer) formed by simple hydration of dried films proved to be stable for at least several weeks, which as a point of reference is a much longer period than the time scale for in vivo circulation of related copolymer vesicles. Thus, stability was not considered to be a problem for in vivo applications. Moreover, internalization of the worm micelles was demonstrated through biotin-receptor endocytosis, as compared with pristine worm micelles that showed little interaction without functionalization. Accordingly, other cell-specific ligands can also be attached to the worm micelles to target specific delivery of encapsulated molecules (e.g., drugs), including micron-long filamentous phages that infiltrate tumors in vivo and specifically bind via displayed peptides.

In addition, Publ. US Pat. Appl. 2005/0180922 confirmed that the micron length, flexible, worm micelles of the present invention, have a significantly longer circulation time than vesicles that had previously been utilized as delivery vehicles, and demonstrate their ability to load and transport a hydrophobic encapsulated material (i.e., drug) to a specific cell receptor. The final copolymer (OEX_{hTfR}) contained a 12 amino acid peptide, which had previously been shown to bind to human transferrin receptor (hTfR) (Lee et al., Eur. J, Biochem. 268:2004 (2001)). hTfR is generally up-regulated on proliferating tumor cells (Miyamoto et al., Int. J. Oral Maxillofac. Surg. 23:430 (1994); Keer et al., J. Urol. 143:381 (1990)). OEX_{hTfR} was blended into the worm micelles at 1% total number of OEX copolymers per worm, which was sufficient to functionalize an aggregate.

Worm micelles used for the in vivo assays were a 10% molar blend of OB3 in OEX, wherein the final copolymer (OEX_{hTfR}) contained the above-identified 12 amino acid peptide that binds to hTfR. In the in vivo assay of the incorporated published application, OEX_{hTfR} was also blended into the worm micelles at 1% total number of OEX copolymers per worm to functionalize an aggregate. The purpose of the assay was to demonstrate the stability of the worm micelles in circulation, and in fact, the worm mass stayed relatively constant over a period of three days until the worm micelles were finally broken down to sub-micron size vesicles, which were then cleared as PEGylated spherical objects. Thus, the large diameter OB18 worm micelles were shown to be stable and less susceptible to fragmentation.

In vitro assays demonstrated that worm micelles can be loaded with a cytotoxic drug (such as paclitaxel), and deliver the drug via a specific targeting peptide to tumor cells expressing selected human receptors by ligand-receptor binding. Consequently, the cylindrical geometry of the stable worm micelles formed from PEG-based diblock copolymer amphiphiles have been shown to provide a useful alternative to spherical carriers that are short-lived in the vasculature of a mammal. When incubated with smooth muscle cells that express a biotin receptor, worm micelles specifically bound to the cell surface and transferred their dye contents. As a result, the worm micelles have a demonstrated utility, not only to encapsulate and deliver active agents, but they have proven their potential for ‘targeted’ drug delivery to specific cell types.

Delivery of one or more active agents to either plants or animals, and particularly to humans, is contemplated in the present invention. Because the administration and use of a variety of drug delivery vehicles, including controlled release vehicles, is well known in the art, one of ordinary skill would know how to select and quantify the drugs or other biocompatible compositions to be delivered to a patient, as well as methods for administering the loaded worm micelles of the present invention to a patient and monitoring the release of the one or more active agents and finally the removal of the fragmented worm micelles from the patient's system.

Adjustments of molecular weight, composition and polymerization of the micelle can be readily adapted to the size and viscosity of the selected drug by one of ordinary skill in the art using standard techniques, and the release of an encapsulated active agent can be controlled by the length of the worm. Once the encapsulated active agent has been released and the worm has been fragmented, it is then quickly removed from the patient's circulation.

In bioremediation, the worm micelles could effectively transport waste products, heavy metals and the like. In electronics, optics or photography, the worm micelles could transport chemicals or dyes. Moreover, these stable micelles may find unlimited mechanical applications including insulation, electronics and engineering. Additional encapsulation applications that involve incorporation of hydrophobic molecules in the bilayer core include, e.g., alkyd paints and biocides (e.g., fungicides or pesticides), obviating the need for organic solvents that may be toxic or flammable. Worm micelles also provide a controlled microenvironment for catalysis or for the segregation of non-compatible materials both in vivo and in vitro.

The present invention is further described in the following examples in which experiments were conducted to characterize the hydrolytic degradation of worm micelles. These examples are provided for purposes of illustration to those skilled in the art, and are not intended to be limiting unless otherwise specified. Moreover, these examples are not to be construed as limiting the scope of the appended claims. Thus, the invention should in no way be construed as being limited to the following examples, but rather, should be construed to encompass any and all variations that become evident as a result of the teaching provided herein.

### EXAMPLE

The ability to control and regulate the hydrolytic shortening (fragmentation) of worm micelles was demonstrated using worm micelles prepared from poly(ethylene oxide)-block-poly(ε-caprolactone) copolymers (PEO-PCL, also denoted OCL). Two PEO-PCLs, with weight fractions of PEO, f_{EO}˜0.42, of different molecular weights were used: OCL1 (Mn=2000-2770, polydispersity PDI=M_{w}/Mₙ=1.19; OCL3 (Mn=5000-6500, polydispersity PDI=M_{w}/Mₙ=1.3.

Worm micelles were produced from each of the PEO-PCLs using a co-solvent/evaporation method. A 100 μl aliquot of a stock solution (10 mg/ml) of OCL1 or OCL3 in chloroform was placed in a glass vial and chloroform was removed under a stream of nitrogen. The OCL film which formed was dissolved in 30 μl chloroform, 5 ml of water was added and the mixture was stirred vigorously for 1-2 hours, yielding an opaque worm micelle dispersion (0.2 mg/ml). Chloroform was slowly removed by evaporation at 4° C. to minimize degradation of the worm micelles. After 24 hours, the worm micelle solution turned clear and did not contain detectable chloroform by gas chromatography (GC), with a detection limit of 0.01% volume fraction of chloroform. The OCL copolymers are mainly in the form of micelles in aqueous solution, since the concentration of OCL worm micelles (0.2 mg/mL) is approximately 100 times the CMC of OCL copolymers, which is around 1.2 μg/ml (Luo et al., Bioconjugate Chem. 13:1259-1265 (2002)), but also exponentially decreasing with PCL chain length.

OCL worm micelles were evaluated over time using three methods: visualization, gel permeation chromatography and nuclear magnetic resonance. Visualization of micelles to determine the contour length distribution and flexibility of OCL worm micelles was performed over time using an Olympus IX71 inverted fluorescence microscope with a 60× objective, and images were recorded using a Cascade CCD camera. A hydrophobic fluorophore dye (PKH 26) was added to the aqueous solution of OCL worm micelles and 2 μl samples of the aqueous solution were placed in a chamber between a glass slide and the cover slip. Approximately 20 pictures were taken of each sample. Contour length distribution and flexibility of OCL worm micelles were obtained from the analysis of more than 150 worm micelles using methods described by Dalhaimer et al., 2003, supra, and by Geng et al., J. Phys. Chem. B 109(9):3772-3779 (2005)).

The amount of 6-hydroxycaproic acid (6-HPA), the monomer formed from the hydrolysis of the caprolactone block in the micelles at various times, was determined using gel permeation chromatography (GPC). At each sampling time a 1 ml aliquot of the 0.2 mg/ml OCL worm micelle solution was lyophilized to a dry powder, re-dissolved in 150 μl tetrahydrofuran (THF), and passed through a 0.4 μm syringe filter. The filtered solution was analyzed on a Waters Breeze GPC equipped with a refractive index detector and a manual injector connected with Styragel HR2 and HR3 columns, using THF as the mobile phase with a flow rate of 1.0 ml/min. Chromatographic peaks corresponding to copolymers were determined using polyethyleneoxide standards of dimer, trimer, tetramer and larger caprolactone oligomers. The predominant new peak formed during shortening of the OCL micelle was identified as 6-hydroxycaprioc acid (6-HPA) based on co-elution of the new peak in the solution with standard 6-HPA (Sigma-Aldrich). The amount of 6-HPA present was determined from a standard calibration curve which correlated peak area with the amount of 6-HPA present.

The loss of caprolactone units from OCL copolymer over time during worm micelle shortening was determined from ¹H nuclear magnetic resonance (NMR). At each sampling time a 20 ml aliquot of the 0.2 mg/ml OCL worm micelle solution was lyophilized after removing degradation products by dialysis (molecular weight cut-off (MWCO) of 2000 and 6000 kD for OCL1 and OCL3, respectively) at 4° C. The dried material was re-dissolved in deuterated chloroform and analyzed on a Bruker 300 MHz spectrometer. The number of caprolactone units (PCLₜ) remaining in the copolymer was estimated by comparing the summation of the integral of the PCLₜ methylene peaks (δ˜4.0, 2.3, 1.6 and 1.3 ppm, total number of protons=10×unit_{PCLt}) to the integral of PEO methylene peak (δ˜3.6 ppm s, total number of protons=4×unit_{PCLt}), which is non-degradable and remains constant during shortening of the OCL worm micelle.

The affect of pH on worm micelle length shortening was evaluated in pH 5 and pH 7 buffers at temperatures of 4, 25 and 37° C. Worm micelles were produced using the cosolvent evaporation method described above, where pH 5 or pH 7 buffers was added to the OCL film to form micelles. Micelle length was evaluated over time using fluorescence microscopy, as described above.

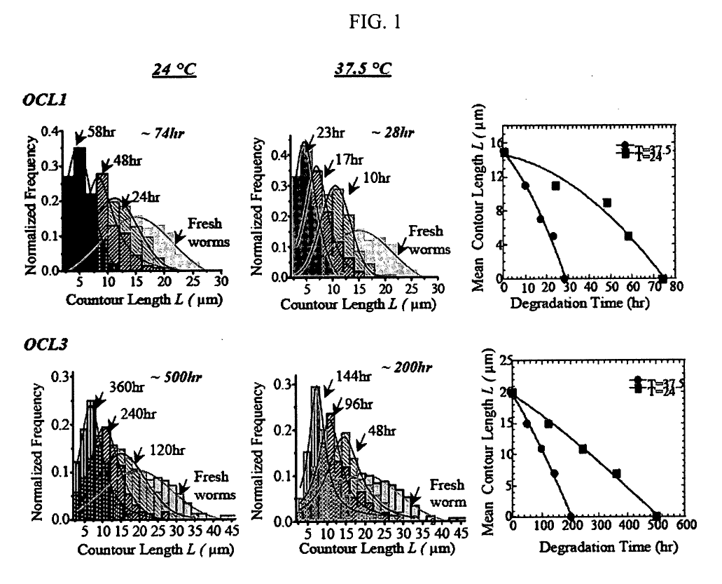

FIG. 1 illustrates the change in contour length distributions over time in worm micelles having different initial molecular weights. Worm micelles from OCL1 (Mn=2000-2770, polydispersity PDI=M_{w}/Mₙ=1.19) required approximately 74 and 28 hours to shrink into spherical micelles at 24 and 37.5° C., respectively, while worm micelles from OCL3 (Mn=5000-6500, polydispersity PDI=M_{w}/Mₙ=1.3) required approximately 500 and 200 hours to shrink into spherical micelles at 24 and 37.5° C., respectively. Higher molecular weight diblock copolymers (OCL3, Mn=5000-6500,) took longer to shrink into spherical micelles than lower molecular weight diblock copolymers (OCL1, Mn=2000-2770), 500 hours versus 74 hours at 24° C. and 200 hours versus 28 hours at 37.5° C. The rate at which worm micelles shrink to become spherical micelles can be modified by changing the molecular weight of the diblock copolymers. In worm micelles constructed of other multi-block copolymers, the rate at which they shrink to become spherical micelles is also changed by modifying the molecular weight of the copolymer. Methods for changing the molecular weight of the multi-block copolymers are know to one skilled in the art.

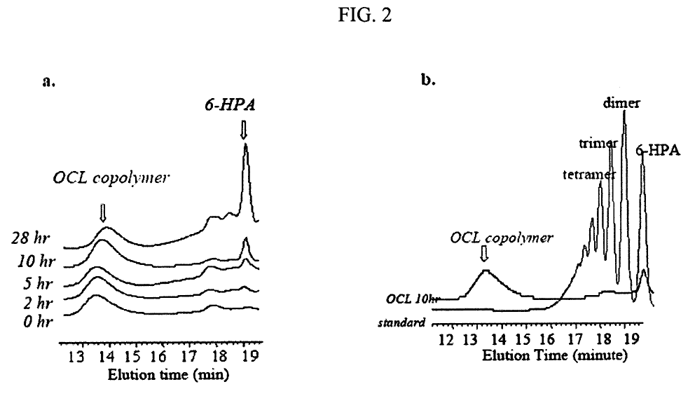

FIG. 2a illustrates the formation of 6-HPA over time from the hydrolysis of OCL1 worm micelles. The amount of 6-HPA, the predominant new peak, increased significantly over time, while no other predominant peak was observed. FIG. 2b illustrates that the new predominant peak formed by hydrolysis is 6-HPA and not a dimer, trimer, tetramer or larger caprolactone oligomers, as shown by GPC analysis. This demonstrates that the PCL in the copolymer hydrolyzes from the end by “chain-end cleavage” to produce 6-HPA rather than by a process of “random scission” that would produce a mixture of various degradation products, including dimers, trimers, tetramers, and larger oligomers. “Chain-end cleavage” of PCL increases the fraction of ethylene oxide, f_{EO}, and consequently shifts the preferred morphology toward a higher curvature structure, from a cylinder toward a sphere (Jain et al., Science 300:460-464 (2003)).

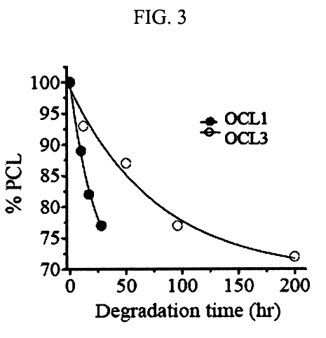

FIG. 3 illustrates the percentage of caprolactone units remaining in worm micelles over time from block copolymers of differing molecular weights by ¹H NMR analysis. Only 75% of the initial OCL1 and OCL3 copolymer remained after approximately 25 and 100 hours, respectively. This indicates that worm micelles with higher molecular weights are hydrolyzed at a slower rate than worm micelles with lower molecular weights. Shrinkage of the PCL chain length reduces the fraction of PCL in the copolymer, which increases the fraction of ethylene oxide in the copolymer, f_{EO}, from 0.42 to 0.55 when the worm micelles disappear and spherical micelles form.

Table 2 shows the time constants (τ) for the shortening of worm micelles at pH 5 and pH 7 buffer at various temperatures.

TABLE 2
Time constants (τ) for the shortening of OCL1 and OCL3
worm micelles in pH 5 and pH 7 buffer at various temperatures
Time constants (τ) in hrs

| | |
|---|---|
| OCL1 | OCL3 |

| Temperature | pH 5 | pH 7 | pH 5 | pH 7 |
|---|---|---|---|---|
| 4° C. | ˜45 | ˜206 | ˜963 | ˜2345 |
| 25° C. | ˜18 | ˜65 | ˜220 | ˜412 |
| 37° C. | ˜10 | ˜30 | ˜108 | ˜108 |

The rate at which worm micelles of differing molecular weights (OCL1 (Mn=2000-2770); OCL3 (Mn=5000-6500) shortened was affected by both temperature and pH. Shrinkage of worm micelles was more rapid at higher temperature and at lower pH. The rate of shrinkage was higher with the lower molecular weight micelles. This demonstrates that the rate of shrinkage of a worm-micelle can be controlled by the composition of the block copolymers as well as the pH and temperature of the environment in which the worm micelles are placed. By controlling the hydrolytic shortening of worm micelles, the delivery and release of materials encapsulated within the worm micelle is controlled.

All patents, patent applications and publications referred to in the present specification are also fully incorporated by reference.

While the foregoing specification has been described with regard to certain preferred embodiments, and many details have been set forth for the purpose of illustration, it will be apparent to those skilled in the art that the invention may be subject to various modifications and additional embodiments, and that certain of the details described herein can be varied considerably without departing from the basic principles of the invention. Such modifications and additional embodiments are also intended to fall within the scope of the appended claims.

What is claimed is:

1. A method of delivering an active agent comprising:
encapsulating said active agent in a worm-like micelle, wherein said worm-like micelle comprises one or more amphiphilic block copolymers capable of self assembly in aqueous solution, and wherein the amphiphilic block copolymer comprises at least one hydrophilic block and at least one hydrophobic block, the at least one hydrophobic block being hydrolytically unstable in the pH range of about 5 to about 7, wherein at least one hydrophobic block is selected which degrades in the micelle at a rate which controls the rate of hydrolysis of the worm-like micelle; and
delivering said encapsulated active agent to a living organism, wherein said hydrophobic block decomposes at a know rate based on a known pH, thereby releasing said active agent.

2. The method of claim 1, wherein said active agent is selected from the group consisting of therapeutic compound, dye, indicator, biocide, nutrient, protein or protein fragment, salt, gene or gene fragment, steroid, and gas.

3. The method of claim 1, wherein the one or more amphiphilic block copolymer of the micelle comprise at least one multi-block copolymer.

4. The method of claim 1, wherein the one or more amphiphilic block co-polymers of the micelle comprise at least one diblock copolymer.

5. The method of claim 1, wherein all of the amphiphilic molecules comprising the micelle are block copolymers.

6. The method of claim 1, wherein the decomposition of the hydrophobic block predominantly forms a degradate of a monomer of the hydrophobic block.

7. The method of claim 1, wherein the ratio of the quantity of hydrophilic block copolymer to the quantity of hydrophobic block copolymer in the micelle ranges from about 0.42 to about 0.55.

8. The method of claim 1, wherein polydispersity is 1.3 or less.

9. The method of claim 1, wherein the hydrophilic block comprises polyalkyleneoxide or polyethylene glycol.

10. The method of claim 1, wherein the hydrophobic block comprises polycaprolactone.

11. The method of claim 1, wherein the hydrophilic block comprises polyethyleneoxide, the hydrophobic block comprises polycaprolactone, and the hydrophilic block comprises a fraction from about 0.42 to about 0.55.

12. The method of claim 1, further comprising controlling the release of the active agent encapsulated in the worm-like micelle by adjusting the ratio of the quantity of said hydroplilic block copolymer to the quantity of said hydrophobic block copolymer in the micelle to control the rate of hydrolysis of said worm-like micelle.

13. The method of claim 1, wherein the ratio of the quantity of the hydrophilic block copolymer to the quantity of the hydrophobic block copolymer in the micelle is adjusted by blending different diblock copolymers.

14. A method of claim 1, further comprising modifying the pH of the environment around the worm-micelle to regulate the controlled degradation of the worm-like micelle.
